(19) United States
(12) Patent Application Publication
(10) Pub. No.: US 2023/0167430 A1
(43) Pub. Date: Jun. 1, 2023

(54) SYSTEMS, DEVICES, AND METHODS FOR ELECTROPHORETIC EXTRACTING AND ENRICHING EXTRACHROMOSOMAL DNA

(71) Applicant: Sage Science, Inc., Beverly, MA (US)

(72) Inventor: T. Christian BOLES, Bedford, MA (US)

(21) Appl. No.: 17/921,049

(22) PCT Filed: Apr. 23, 2021
(86) PCT No.: PCT/US2021/028922

§ 371 Date: Oct. 24, 2022

Related U.S. Application Data
(60) Provisional application No. 63/015,288, filed on Apr. 24, 2020.

(51) Int. Cl. C12N 15/10; C12Q 1/6806; G01N 27/447

(52) U.S. Cl. CPC C12N 15/101; C12Q 1/6806; G01N 27/44704; G01N 2030/8827

(57) ABSTRACT

Embodiments of the present disclosure present methods, systems, and devices for extrachromosomal DNA extraction, and in some embodiments, isolation of DNA therefrom, and/or analysis of the extracted and/or isolated DNA, including, in some embodiments, ecDNA.

### RELATED APPLICATIONS

This application claims benefit of and priority to U.S. provisional patent application No. 63/015,288, filed Apr. 24, 2020, the entire disclosure of which is herein incorporated by reference.

### BACKGROUND

In many procaryotic and eucaryotic organisms, the DNA comprising the genetic makeup of the cell and its organelles can differ dramatically in size and topology. In mammals, the nuclear chromosomal DNA is linear and ranges in size from tens to hundreds of megabases (Mbs). In addition to chromosomal DNA, each mammalian cell carries, generally, several thousands of copies of a circular 16 kilobase (kb) DNA per cell within their mitochondria (Vetri et al., 1990).

Recent studies have shown that eucaryotic cells also contain extrachromosomal DNA (ecDNA) circles derived from nuclear DNA. In normal cells, these DNAs are small, generally less than 20 kb in size, and are frequently related to repeated chromosomal sequences (Moller, et al., 2018). However, in cancer cells, larger circular ecDNAs, ranging in size from 10's of kb's in size to several Mb's in size, that carry amplified oncogenes or drug resistance genes, can also be found (reviewed in Verhaak, et al., 2019).

Similarly, in bacterial cells, there is frequently a large circular chromosome with an average size of about 4 Mb (chromosome sizes of bacteria range from low hundred kb's to low teens of Mb's) and bacteria also carry smaller circular ecDNAs, commonly termed plasmids (Francia et al., 2004; Sherratt, 1974). These plasmids typically range in size from single kb's to low hundreds of kb's in size. In pathogenic bacteria, such plasmids frequently carry genes that influence virulence, host range, and drug resistance (Pilla and Tang, 2018; Rozwandowicz et al., 2018).

In both bacteria and mammalian cells, extrachromosomal DNA usually comprises a small fraction of the total cellular DNA. Currently, most molecular characterizations of extrachromosomal DNA is carried out bioinformatically using whole genome sequence data and de novo sequence assembly algorithms. Using high coverage whole genome sequence data, it is frequently possible to assemble ecDNA sequences into a single circular contig. However, when the ecDNAs are closely related to chromosomal DNA sequences, and if they have significant levels of rearrangement, assembly of ecDNAs can be difficult (Wu et al., 2019).

Since ecDNAs play important roles in human disease, it is important to develop new, cost effective methods for analyzing them.

### SUMMARY

Embodiments of the present disclosure present methods, systems, and devices for extrachromosomal DNA extraction, and in some embodiments, isolation of DNA therefrom, and/or analysis of the extracted and/or isolated DNA.

Accordingly, in some embodiments, an extrachromosomal DNA (ecDNA) extraction and isolation method is provided which includes providing an agarose gel column configured for DNA electrophoresis, the gel column configured to include or be contained in at least two compartments (which may also be referred to throughout the present disclosure as cavities or wells), depositing a sample comprising a cell suspension comprising a plurality of cells within a first compartment of the at least two compai intents that is arranged proximal to a first, positively charged electrode, the positively charged electrode configured to attract a negatively charged detergent and DNA during electrophoresis, depositing a lysis reagent comprising at least one negatively-charged detergent within a second compartment of the at least two compartments, the second compartment arranged proximal to a second, negatively charged electrode, applying a first electrophoretic field via the first and second electrodes such that the negatively charged detergent moves to and into or through the first compartment containing the cell suspension, such that cells in the in the first compartment are lysed substantially without any viscous shear from liquid mixing, applying a second electrophoretic field or continuing the first electrophoretic field so as to conduct electrophoresis under conditions suitable for size selection of desired ecDNA, such that the ecDNA is separated from larger chromosomal DNA molecules and travels down the gel column, and isolating the size selected ecDNA from the gel column.

Such embodiments can include one and/or another of (and in some embodiments, a plurality of, and in some embodiments, a majority of, and in still other embodiments, substantially all, or all of) the following features, functions, structure, steps, processes, objectives, advantages, and clarifications, yielding yet further embodiments of the present disclosure:
electrophoresis results in DNA of a size greater than 3 Mb become immobilized in the agarose gel;
electrophoresis results in the DNA of greater than 3 Mb being immobilized in the wall of the first compartment;
electrophoresis results in DNA having a size of less than 3 Mb traveling down the gel column;
the DNA having a size of less than 3 Mb is isolated via electroelution;
electroelution results in size fractions of the DNA less than 3 Mb in size being eluted to one or more elution modules of an elution cassette;
the DNA of less than 3 Mb comprises ecDNA;
a time period to complete electrophoresis corresponds to the size of the ecDNA;
electrophoresis is performed between 2 and 9 hours;
analyzing the ecDNA;
analyzing at least one characteristic of the isolated ecDNA, where the at least one characteristic can be selected from the group consisting of size, topology, and sequence content;
enriching the ecDNA;
the plurality of cells comprise animal cells;
the plurality of cells comprise mammalian cells;
the plurality of cells comprise human cells;
the plurality of cells comprise fungal cells;
the plurality of cells comprise fungal cells that have been enzymatically treated to remove cell walls;
the plurality of cells comprise plants cells;
the plurality of cells comprise plants cells that have been enzymatically treated to remove cell walls which would otherwise prevent cell lysis by anionic detergents;
the plurality of cells comprise bacterial cells;
the plurality of cells comprise bacterial cells that have been enzymatically treated to remove cell walls which would otherwise prevent cell lysis by anionic detergents;
the cells in the cells suspension are uniformly dispersed;
the at least two compartments are arranged proximate one another;
isolating the size selected ecDNA from the gel column is via electroelution;
the second electrophoretic field and/or continuing the first electrophoretic field is applied so as to conduct electrophoresis under conditions suitable for size selection of desired ecDNA, such that the ecDNA is separated from larger chromosomal DNA molecules;
isolating the size selected ecDNA from the gel column is via electroelution;
determining the DNA sequence of the ecDNA;

and
imaging the ecDNA, where imaging can be via at least one of: optically, electron microscopy, atomic force microscopy.

An agarose gel cassette and/or system including at least two wells/cavities/compartments, for holding liquid samples positioned in relatively close proximity and configured to enable or perform one or methods of the present disclosure.

These and other embodiments, advantages, and objects of the disclosure will become even more evident with reference to the accompanying figures, a brief description of which is provided below, and detailed description which follows.

### BRIEF DESCRIPTION OF THE DRAWINGS

FIG. 1a is a perspective, exploded view of an agarose gel cassette, according to some embodiments of the disclosure;

FIG. 1b is a perspective view of an agarose gel cassette of FIG. 1a, according to some embodiments of the disclosure;

FIG. 2a illustrates a top view of an assembled cassette, with a top cover, according to some embodiments of the disclosure;

FIG. 2b illustrates a top view of the assembled cassette of FIG. 2a with the top cover removed, according to some embodiments of the disclosure;

FIG. 3 is a schematic view of a method according to some embodiments of the present disclosure;

FIGS. 4a and 4b are graphs illustrating results of qPCR (with respect to Example 1), according to some embodiments of the present disclosure;

FIG. 5 is a result graph illustrating mtDNA extraction in performing methods according to some embodiments of the disclosure;

FIGS. 6-7 are result charts illustrating Miseq mtDNA coverage, reads with respect to certain genes.

### DETAILED DESCRIPTION

Embodiments of the present disclosure are directed to methods, systems, and devices for extracting ecDNA (and in some embodiments, enriching isolated/extracted ecDNA), while separating ecDNA from chromosomal DNA for downstream molecular analysis by, for example, DNA sequencing, and imaging via, for example, optical mapping, electron microscopy, and atomic force microscopy.

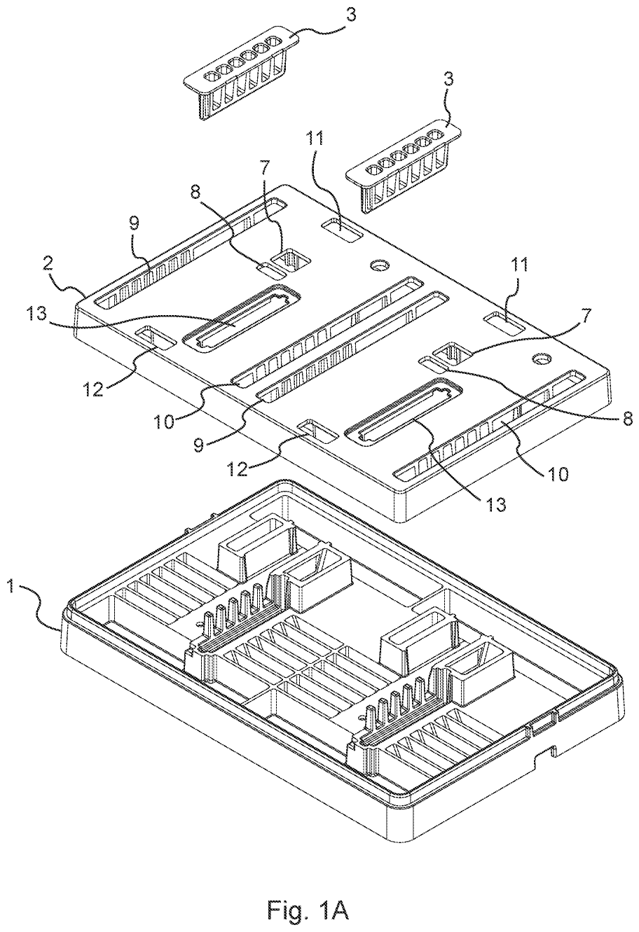

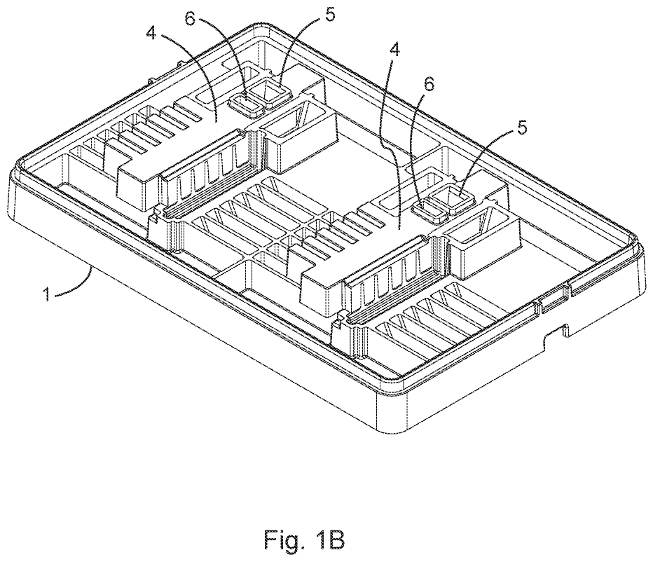

In some embodiments, the systems and/or devices upon which methods of the disclosure can be performed (or performed with) include, inter alia, gel electrophoresis instruments and consumables of the general form, e.g., illustrated in FIGS. 11-13, and FIG. 15 of U.S. Ser. No. 10/131,901B2, and related patents and applications (all incorporated herein by reference in their entities). In some embodiments, and as shown in FIGS. 1a-b, the consumable is an agarose gel cassette with two wells/cavities/compartments (such terms used interchangeably in the present disclosure), for holding liquid samples positioned in relatively close proximity (preferably 1-5 mm apart, more preferably 1-3 mm apart). The gel is formed in a buffer suitable for DNA electrophoresis. The gel is preferably from 0.3% to 3% in agarose concentration (weight % grams per 100 ml), and more preferably from 0.75% to 1.5%.

Accordingly, in FIG. 1a, which is an exploded view of a cassette (according to some embodiments), including a top (1), a bottom (2), and an elution module assembly (3). The cassette top and bottom can be glued or otherwise affixed together. A left face of the elution module can be bonded to a DNA-permeable filter material, and a right face can be bonded to a DNA-impermeable ultrafiltration membrane. After assembly of cassette top and bottom, the elution module can be glued or otherwise affixed into slot (13) in the cassette top. After insertion into the cassette, the left face of the elution module is configured to form a right border of the gel column.

Each cassette can include two independent sample processing zones, separated left and right by a wall which extends along from the inside of the cassette top (2) between the elution electrode channels (9) and (10) located in the central region of the cassette (not visible in the view). The cassette top includes ports for negative separation electrodes (11), and ports for positive separation electrodes (12). Separation electrodes are configured to provide an electrophoretic field that moves negatively charged molecules along the gel column axis. Also shown are ports for negative elution electrodes (9), and positive elution electrodes (10), which may be used to electroelute negatively charged molecules out of the gel from left to right into the buffer-filled elution modules. The ports for the sample well (8) and reagent well (7) are also indicated.

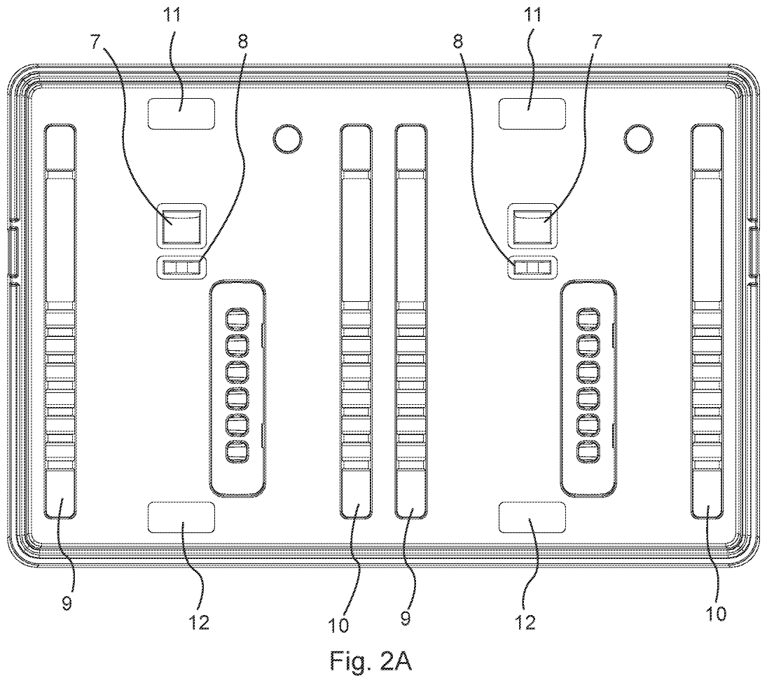

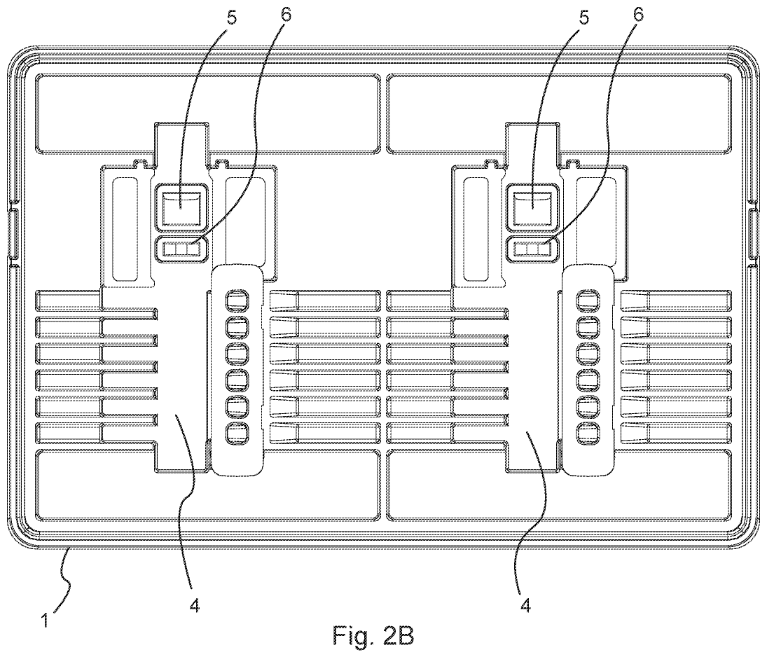

FIG. 1b illustrates a position of the gel columns (4) in the cassette with the cassette top removed. In some embodiments, and as shown, the gel appears to be unsupported on several sides, because its boundaries are defined by walls on the underside of the cassette top. The positions of the sample wells (6) and reagent wells (5) are indicated. FIG. 2a illustrates a top view of the assembled cassette, while FIG. 2b illustrates a top view of the assembled cassette with the top cover removed. All labels are the same as for FIGS. 1a and 1b.

In some embodiments, suitable samples can be uniform cell suspensions that can be lysed by an anionic (negatively charged) detergent such as sodium dodecyl sulfate. Eucaryotic cells without cell walls are examples of suitable cells. Bacterial, fungal, and plant cells can be used as samples if the cells are treated with appropriate enzymes that will degrade their cell walls prior to their use with the extraction method according to the present disclosure.

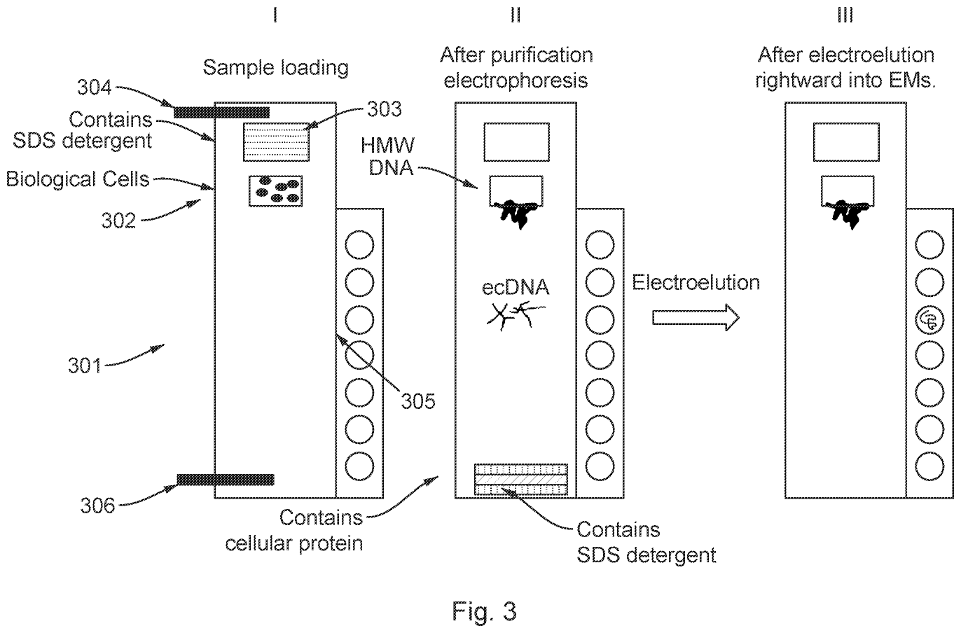

A schematic view of a method according to some embodiments is illustrated in FIG. 3, with a block representation of a cassette (300)/elution module (305) for performing the method (e.g., see FIGS. 1a-2b). Please note, the identifying numbers in “I” of FIG. 3 apply to to “II” and “III” of FIG. 3. Accordingly, a sample comprising a uniformly dispersed cell suspension is placed in a well (302) that is proximal to a positively charged electrode (306) (i.e., a cavity toward which the negatively charged detergent and DNA travel during electrophoresis). A lysis reagent comprising at least one negatively-charged detergent is placed in a well (303) that is proximal to a negatively charged electrode (304). An electric field is then applied via the electrodes so that the negatively charged detergent moves out of well (303) and into/through the sample well (302) that contains the cell suspension, such that, the cells in the sample well are lysed gently without any viscous shear from liquid mixing. Small cellular components and nucleases are denatured, coated by the detergent, and are carried toward the positive electrode (306). Accordingly, in some embodiments, cellular DNA larger than approximately 3-4 million base pairs (Mb) in length is driven into the sample well wall (see “HMW DNA” in “II”, i.e., the central portion of FIG. 3), but quickly becomes entangled in the gel of/near the well wall and immobilized there. Smaller DNA molecules (<approximately 3-4 Mb), including ecDNA, however, do not become immobilized in the sample well wall, and travel down the gel column (301)(see, e.g., “ecDNA” in “II”). The smaller ecDNA can be size-separated in the gel using appropriate gel electrophoresis conditions, and electroeluted from the gel into buffer-filled elution modules (see “III” of FIG. 3) of that are positioned adjacent to the gel column (e.g., using apparatus based on that cited in U.S. Ser. No. 10/131,901 B2; such apparatus is commercially available as the SageHLS system, Sage Science, Inc., Beverly, Mass.; https://sagescience.com/products/sagehls/).

After elution, the ecDNA products can be recovered from the elution modules using standard manual or automated liquid handling methods. If the DNA is >50 kb in size, wide-bore pipette tips and slow pipetting speeds are suggested so as to avoid shear breakage of the products. This electrophoretic ecDNA purification method (according to some embodiments) is simple and fast, especially compared with non-electrophoretic methods discussed in the previous section (in some embodiments, 2-9 hrs. total electrophoresis time depending on the size of the ecDNA).

Exemplary detergents for the lysis reagent, according to some embodiments, include sodium dodecyl sulfate (SDS) and sodium N-lauroyl sarcosinate (sarkosyl) at concentrations between about 0.1% and about 10% (w/v). One preferable lysis reagent is SDS at a concentration between 2% and 5%.

After recovery, the enriched ecDNA products can be analyzed by different methods including quantitative PCR, DNA sequencing, optical imaging, electron microscopy (EM), and atomic force microscopy (AFM). Quantitative PCR is useful for identifying the copy number and elution position of specific known sequence elements carried on the ecDNAs. The elution position under a given set of electrophoresis conditions will also provide some estimates on the size of the ecDNA identified by qPCR. Optical imaging, EM, and AFM, can provide more direct measurements of the size and topology of ecDNAs (Cai et al., 1998; Boles et al., 1990; Mikheikin et al., 2017). In addition, optical imaging and AFM methods can also provide long-range maps of specific DNA sequence elements, which can provide useful scaffolds for checking and correcting ecDNA sequence data (Cai et al., 1998; Wu et al., 2017; Mikheikin et al., 2017).

Enriched ecDNA products can be sequenced by all standard sequencing methods, including short-read Illumina paired-end sequencing, long-read methods such as PacBio or Oxford Nanopore sequencing, or combination approaches such as linked-read sequencing (10× Genomics, Universal Sequencing Technologies' TELL-seq, Chen et al., 2019).

Enriched ecDNAs produced by the method, according to some embodiments, can be contaminated by low amounts of SDS which are difficult to completely remove from the gel during extraction, and which co-elute with the ecDNA. Small ecDNAs (less than approximately 20 kb) can be purified using solid-phase reversible immobilization methods (DeAngelis et al., 1995) to remove SDS. Longer ecDNAs can be broken by the SPRI method, and in such cases, it may be preferable to concentrate DNA and remove SDS by ethanol precipitation, optionally with inert carrier polymers (glycogen or linear polyacrylamide) to ensure efficient precipitation of low amounts of ecDNA (Fregel et al., 2010).

### EXAMPLES

In the examples, reference is made to electrophoresis buffer K, which is used at 0.5× strength in the gel and reservoir buffers of the SageHLS cassettes. 1×K buffer is 102 mM Tris base, 57 mM N-[Tris(hydroxymethyl)methyl]-3-aminopropanesulfonic acid, [(2-Hydroxy-1,1-bis(hydroxymethyl)ethyl)amino]-1-propanesulfonic acid (TAPS), 0.16 mM EDTA acid, pH8.7.

### Example 1: Extraction and Purification of Plasmids from E. coli W (ATCC 9637)

E. coli W carries two plasmids, pRK1 (102,536 bp) and pRK2 (5,360 bp) (Archer et al., 2011). Fresh overnight cultures of E. coli W were prepared in LB broth with shaking at 37 C. Cell were washed two times by resuspension and centrifugation (12,000×g, 2 minutes) in Wash buffer (10 mM, Tris-HCl pH7.5, 5 mM EDTA, 20% sucrose wt/v), and re-suspended in Spheroplast buffer (10 mM Tris-HCl pH7.5, 5 mM EDTA, 100 mM NaCl, 20% sucrose). Cells were then digested with Ready-Lyse lysozyme (Epicentre/Lucigen), at a final reaction concentration of 5600 units per ml for 30 min at room temperature.

The total DNA concentration of the spheroplast suspension was determined by a rapid SDS lysis procedure. Duplicate aliquots were processed as follows. Ten microliters of sample were mixed with immediate vigorous mixing with 200 microliters of Q lysis buffer (0.5×K buffer, 1% SDS, 5 mM EDTA, 50 mM NaCl). The resulting lysate was diluted with 600 microliters of TE buffer (10 mM Tris-HCl pH8, 1 mM EDTA), and vigorously vortexed for at least 30 seconds. 5 ul aliquots of this final were assayed for DNA content using the Qubit HS reagent kit (Thermo Invitrogen).

To perform the ecDNA extraction, the E. coli W spheroplasts were diluted with Wash buffer to a final DNA concentration of 2.5 micrograms per 70 microliters. A 70 ul aliquot of the diluted spheroplast prep was loaded in the sample well of a 0.75% agarose SageHLS cassette. A 200 microliter aliquot of HLS Lysis buffer (lx K buffer, 2% glycerol (wt/v), 3% SDS, 10 mM EDTA) was loaded into the reagent well, the well ports were sealed with tape, and extraction electrophoresis was initiated immediately.

Two different extraction electrophoresis conditions were used because of the large difference in size between the two plasmids. In the procedure designed to enrich the smaller 5.3 kb plasmid, extraction electrophoresis was carried out for 30 minutes at 50V followed by electroelution laterally at 50 V for 45 minutes. For the larger 102 kb plasmid, extraction electrophoresis was performed using a pulsed field program for 8 hours at 55 V followed by electroelution laterally for 1.5 hours at 50V. The pulsed field program used forward and reverse pulse periods that were linearly incremented for 24 pulsed field cycles. The initial values were 3 seconds forward, and 1 second reverse; in each subsequent F-R cycle the forward pulse was incremented by 2.55 second, and the reverse pulse was incremented by 0.85 seconds. Incrementing was continued for 24 F-R cycles and then the pulse times were returned to their initial conditions (3 seconds F, 1 second R) and the incremention cycle was restarted.

After completion of electrophoresis the elution products were assayed by SYBR green qPCR for the plasmids. To assay for chromosomal DNA in the elution products, qPCR assays for the recA gene were also performed. To suppress PCR inhibition caused by low amounts of SDS in the elution products, a non-ionic detergent hydroxypropyl beta cyclodextrin (bCD) that binds SDS was included in the reactions at 0.1% (wt/v). qPCR reactions (20 microliters) contained SYBR green master mix (PowerUp SYBR Green Master Mix, Thermo ABI), 0.5 micromolar each primer, 0.1% bCD, and 2 microliters of elution product DNA. qPCR was carried out on a QuantStudio 3 instrument (Thermo ABI) using standard SYBR Green conditions (10 minute initial hold at 95 C, followed by forty cycles of 95 C for 15 seconds, 60 C for 1 min). The primers used were:

pRK1:
trbA-1F
TCTTTCCAGGACGTTAAAGG
trbA-1R
GTCGAACAGCATACTCTCAT
pRK2:
mobA-1F
GAAAATGCTGAACGACGAAT
mobA-1R
GATTTTCGTCTCGTTTGAGG
recA:
recA-1F
TATCAACTTCTACGGCGAAC
recA-1R
CTTTACCCTGACCGATCTTC

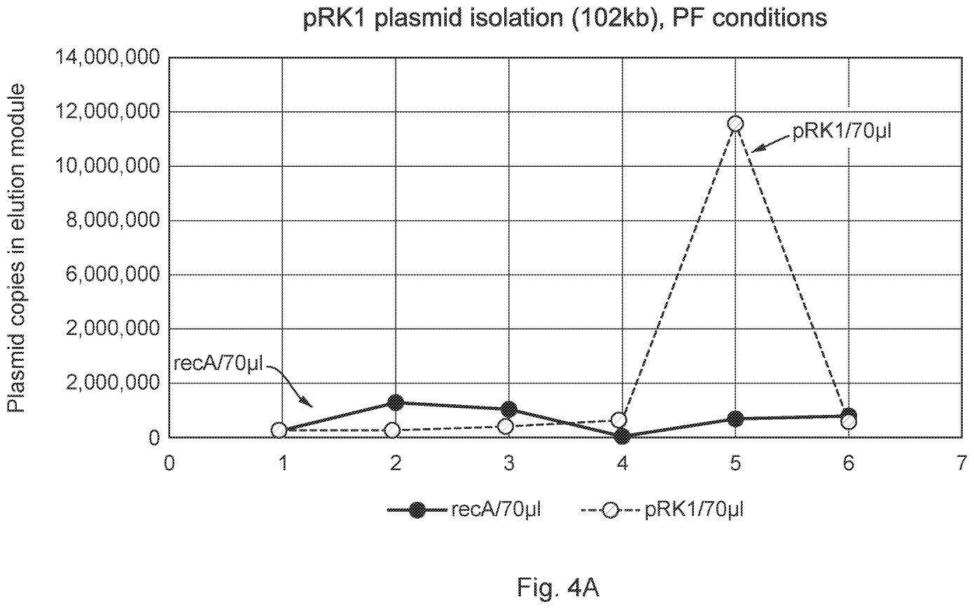

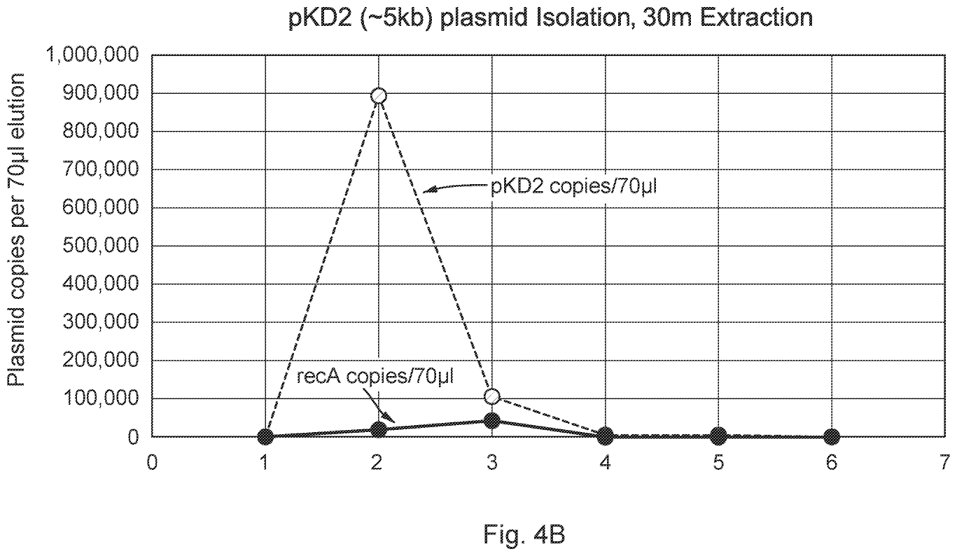

The qPCR results are shown in FIGS. 4a and 4b. FIG. 4a shows that the large pRK1 plasmid (102 kb) eluted in elution fraction 5 of the SageHLS cassette under the pulsed field conditions used. As shown in FIG. 4b, the smaller plasmid was found in elution fraction 2 using the very brief 30 minute extraction electrophoresis period. In both extractions, very little chromosomal DNA was found in the fractions contain plasmid (pRK1/recA copy ratio was 16; pRK2/recA copy ratio was 50), demonstrating enrichment of plasmid DNA.

### Example 2: Extraction and Enrichment of Mitochondrial DNA (mtDNA) from Human White Blood Cells (WBCs)

Human white blood cells (WBC) were isolated from ACD whole blood samples by three centrifugal washes in Red Cell Lysis Buffer (155 mM NH4C1, 10 mM NaHCO₃, 10 mM EDTA), and re-suspended in Suspension buffer (8% sucrose wt/v, 10 mM EDTA, 15% Ficoll400 wt/v, 0.25×K buffer). WBCs were quantified from genomic DNA content using a rapid SDS lysis procedure as described in Example 1 above, followed by DNA quantification using the Qubit HS kit (Thermo Life Invitrogen). For mtDNA extraction, the sample well was loaded with 1.2 million WBCs in 70 ul of Suspension buffer. The HLS workflow included 1.25 hours of extraction and size selection at 50V electrophoresis followed by 1.5 hours of electroelution at 50V to collect the mtDNA into the elution modules of the HLS cassette. The elution position of the mtDNA product was determined by qPCR (Thermo Life ABI Taqman Gene Expression Assay ID: Hs0259874-gl for gene MT-ND2, ABI QuantStudio 3 instrument). As shown in FIG. 3, the mtDNA eluted in fractions 3 and 4. Very little chromosomal DNA was eluted in any fraction and the apparent mtDNA enrichment was >1000-fold by qPCR.

### Example 3: Sequencing of ecDNAs Extracted by the Inventive Method

mtDNA from two aliquots of human WBCs were extracted as described in Example 2 above. qPCR was carried out to find the elution fractions containing mtDNA. Elution product from one lane were used for Oxford Nanopore sequencing on a Minion. Elution products from the other lane were used for paired-end sequencing on an Illumina Miseq.

For both libraries, the HLS elution fraction 4 DNA contained approximately 0.7 nanograms of total DNA (˜80 ul total volume). The DNA was concentrated by ethanol precipitation.

Illumina sequencing libraries were generated with Nextera Flex kits and sequenced using the Miseq 2×150 bp paired end protocol. Illumina short read data was aligned to the hg38 reference genome (see, ftp://ftp.ncbi.nlm.nih.gov/genomes/all/GCA/000/001/405/GCA 000001405.15 GRCh38/s eqs for alignment pipelines.ucsc ids/CA 000001405.15 GRCh38 no alt analysis se t.fna.gz) by BWA-MEM (vs. 0.7.17-r1188, https://github.com/lh3/bwa), and sorted and duplicated using Samtools (vs. 1.9, https://github.com/samtools/samtools). Coverage over mtDNA was evaluated visually with IGV (see, Linux vs. 2.6.2, https://software.broadinstitute.org/software/igv/).

Oxford Nanopore Minion sequencing was carried out using the Rapid Library kit (RAD004) with a Minion R9.4.1 flow cell. For Minion library construction, 50 ng of HMW E. coli genomic DNA was added to the mtDNA-enriched HLS elution product to act as a carrier during library construction. Oxford Nanopore Minion data were base-called post-run in high accuracy mode using guppy software (Oxford Nanopore), and the resulting fastq data file was aligned to the hg38 reference using minimap2 (vs. 2.17_x64-linux, https://github.com/lh3/minimap2) and sorted with Samtools. Read length distributions were analyzed using NanoPlot (vs. L24.0, https://github.com/wdecoster/NanoPlot), and coverage was evaluated visually using IGV.

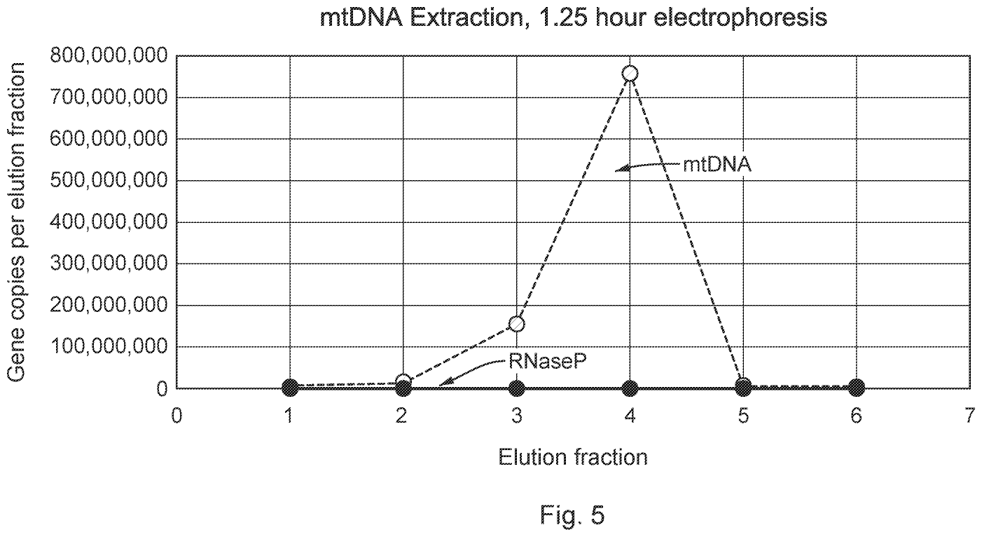

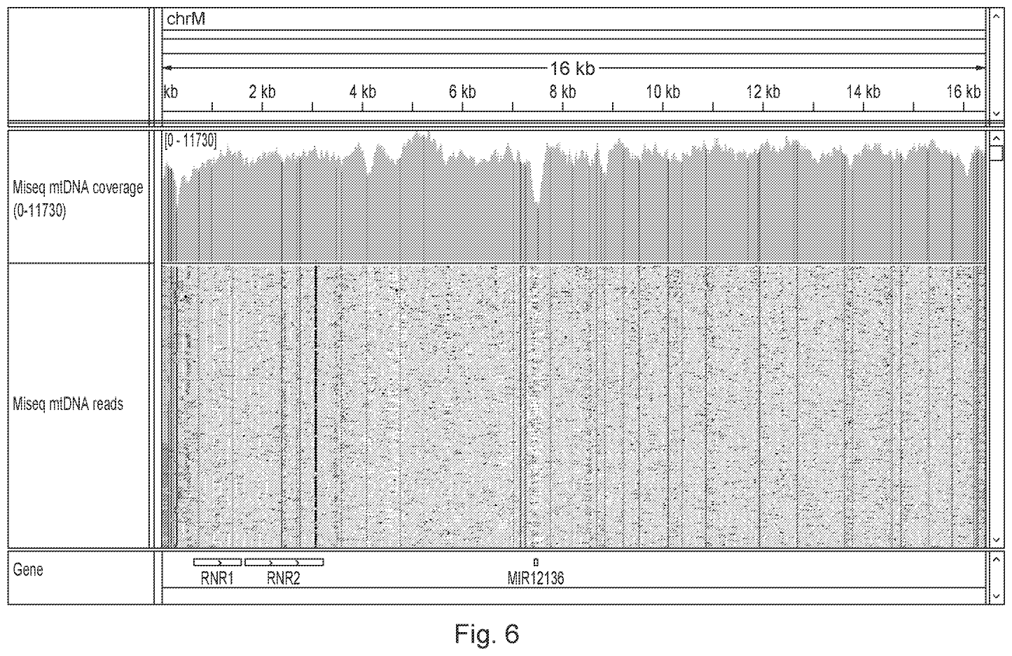

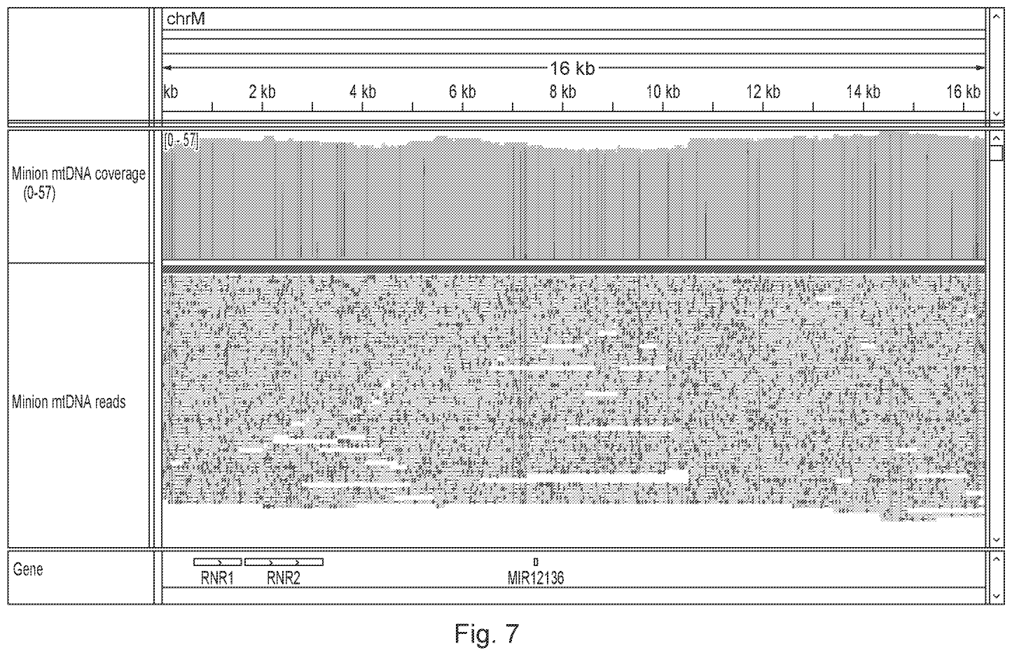

Coverage for Minion sequencing was approximately 60-fold (FIG. 5), and coverage for the Miseq runs was approximately 12,000-fold (FIG. 4b). The Minion coverage was significantly more even over the entire mtDNA genome, as expected from a non-amplified long-read library. The transposase-generated Rapid library had an N50 read length of 15,940 bp, and approximately 50% of the reads were nearly full-length 16,500 reads, apparently resulting from a single transposase insertion into an intact mtDNA circle. Base calling was significantly noisier in the Minion run than in the Miseq. Whereas most of the SNPs identified in the Miseq data could be seen in the Minion data, the Minion data had significantly more potential SNPs, and more sites with multiple base calls. See, e.g., FIGS. 6 and 7.

### Other Considerations for the Disclosure

While various inventive embodiments have been described and illustrated herein, those of ordinary skill in the art will readily envision a variety of other means, functionality, steps, and/or structures (including software code) for performing the functionality disclosed and/or obtaining the results and/or one or more of the advantages described herein, and each of such variations and/or modifications is deemed to be within the scope of the inventive embodiments described herein. More generally, those skilled in the art will readily appreciate that all parameters, and configurations described herein are meant to be exemplary and that the actual parameters, and configurations will depend upon the specific application or applications for which the inventive teachings is/are used. Those skilled in the art will recognize, or be able to ascertain using no more than routine experimentation, many equivalents to the specific inventive embodiments described herein. It is therefore to be understood that the foregoing embodiments are presented by way of example only and that, within the scope of any claims supported by this disclosure and equivalents thereto, inventive embodiments may be practiced otherwise than as specifically described and claimed. Inventive embodiments of the present disclosure are also directed to any and each individual feature, structure, system, apparatus, device, step, code, functionality and method described herein. In addition, any combination of two or more such features, structures, systems, apparatuses, devices, steps, code, functionalities, and methods, if any such combination of features, structures, systems, apparatuses, devices, steps, code, functionalities, and methods are not mutually inconsistent, is included within the inventive scope of the present disclosure. Further embodiments may be patentable over prior art by specifically lacking one or more features, structures, steps and/or functionalities (i.e., claims directed to such embodiments may include one or more negative limitations to distinguish such claims from prior art).

The above-described embodiments of the present disclosure can be implemented in any of numerous ways. For example, some embodiments may be implemented (e.g., as noted) using hardware, software or a combination thereof (e.g., in controlling equipment to carry out one or more steps of disclosed processes). When any aspect of an embodiment is implemented at least in part in software, the software code can be executed on any suitable processor or collection of processors, servers, and the like, whether provided in a single computer or distributed among multiple computers.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to), according to the respective embodiment(s) noted.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### REFERENCES

Veltri K L, Espiritu M, Singh G. Distinct genomic copy number in mitochondria of different mammalian organs. J Cell Physiol. 1990 April; 143(1):160-4. doi: 10.1002/jcp.1041430122. PMID: 2318903.
Møller H D, Mohiyuddin M, Prada-Luengo I, Sailani M R, Halling J F, Plomgaard P, Maretty L, Hansen A J, Snyder M P, Pilegaard H, Lam H Y K, Regenberg B. Circular DNA elements of chromosomal origin are common in healthy human somatic tissue. Nat Commun. 2018 Mar. 14; 9(1):1069. doi: 10.1038/s41467-018-03369-8. PMID: 29540679; PMCID: PMC5852086.
Verhaak R G W, Bafna V, Mischel P S. Extrachromosomal oncogene amplification in tumour pathogenesis and evolution. Nat Rev Cancer. 2019; 19(5):283-288. doi:10.1038/s41568-019-0128-6.
Pilla G, Tang C M. Going around in circles: virulence plasmids in enteric pathogens. Nat Rev Microbiol. 2018 August; 16(8):484-495. doi: 10.1038/s41579-018-0031-2. PMID: 29855597.
Rozwandowicz M, Brouwer M S M, Fischer J, Wagenaar J A, Gonzalez-Zorn B, Guerra B, Mevius D J, Hordijk J. Plasmids carrying antimicrobial resistance genes in Enterobacteriaceae. J Antimicrob Chemother. 2018 May 1; 73(5):1121-1137. doi: 10.1093/jac/dkx488. PMID: 29370371.
Wu S, Turner K M, Nguyen N, Raviram R, Erb M, Santini J, Luebeck J, Rajkumar U, Diao Y, Li B, Zhang W, Jameson N, Corces M R, Granja J M, Chen X, Coruh C, Abnousi A, Houston J, Ye Z, Hu R, Yu M, Kim H, Law J A, Verhaak R G W, Hu M, Furnari F B, Chang H Y, Ren B, Bafna V, Mischel P S. Circular ecDNA promotes accessible chromatin and high oncogene expression. Nature. 2019 November; 575(7784):699-703. doi: 10.1038/s41586-019-1763-5. Epub 2019 Nov. 20. PMID: 31748743; PMCID: PMC7094777.
Cai W, Jing J, Irvin B, Ohler L, Rose E, Shizuya H, Kim U J, Simon M, Anantharaman T, Mishra B, Schwartz D C. High-resolution restriction maps of bacterial artificial chromosomes constructed by optical mapping. Proc Natl Acad Sci USA. 1998 Mar. 31; 95(7):3390-5. doi: 10.1073/pnas.95.7.3390. PMID: 9520376; PMCID: PMC19846.
Boles T C, White J H, Cozzarelli N R. Structure of plectonemically supercoiled DNA. J Mol Biol. 1990 Jun. 20; 213(4):931-51. doi: 10.1016/S0022-2836(05)80272-4. PMID: 2359128.
Mikheikin A, Olsen A, Leslie K, Russell-Pavier F, Yacoot A, Picco L, Payton O, Toor
A, Chesney A, Gimzewski J K, Mishra B, Reed J. DNA nanomapping using CRISPR-Cas9 as a programmable nanoparticle. Nat Commun. 2017 Nov. 21; 8(1):1665. doi: 10.1038/s41467-017-01891-9. PMID: 29162844; PMCID: PMC5698298.
Chen Zhoutao, Long Pham, Tsai-Chin Wu, Guoya Mo, Yu Xia, Peter Chang, Devin Porter, Tan Phan, Huu Che, Hao Tran, Vikas Bansal, Justin Shaffer, Pedro Belda-Ferre, Greg Humphrey, Rob Knight, Pavel Pevzner, Son Pham, Yong Wang, Ming Lei. Ultra-low input single tube linked-read library method enables short-read NGS systems to generate highly accurate and economical long-range sequencing information for de novo genome assembly and haplotype phasing. bioRxiv 852947; doi: https://doi.org/10.1101/852947.
DeAngelis M M, Wang D G, Hawkins T L. Solid-phase reversible immobilization for the isolation of PCR products. Nucleic Acids Res. 1995 Nov. 25; 23(22):4742-3. doi: 10.1093/nar/23.22.4742. PMID: 8524672; PMCID: PMC307455.
Fregel, R., González, A. and Cabrera, V. M. (2010), Improved ethanol precipitation of DNA. ELECTROPHORESIS, 31: 1350-1352. doi:10.1002/elps.200900721.
Archer C T, Kim J F, Jeong H, Park R I, Vickers C E, Lee S Y, Nielsen L K. The genome sequence of E. coli W (ATCC 9637): comparative genome analysis and an improved genome-scale reconstruction of E. coli. BMC Genomics. 2011 Jan. 6; 12:9. doi: 10.1186/1471-2164-12-9. PMID: 21208457; PMCID: PMC3032704.
Francia M V, Varsaki A, Garcillán-Barcia M P, Latorre A, Drainas C, de la Cruz F. A classification scheme for mobilization regions of bacterial plasmids. FEMS Microbiol Rev. 2004 February; 28(1):79-100. doi: 10.1016/j.femsre.2003.09.001. PMID: 14975531.
Sherratt D J. Bacterial plasmids. Cell. 1974 November; 3(3):189-95. doi: 10.1016/0092-8674(74)90130-5. PMID: 4373170

What is currently claimed:

1. An extrachromosomal DNA (ecDNA) extraction and isolation method comprising:
providing an agarose gel column configured for DNA electrophoresis, the gel column configured to include or be contained in at least two compartments;
depositing a sample comprising a cell suspension comprising a plurality of cells within a first compartment of the at least two compartments that is arranged proximal to a first, positively charged electrode, the positively charged electrode configured to attract a negatively charged detergent and DNA during electrophoresis;
depositing a lysis reagent comprising at least one negatively-charged detergent within a second compartment of the at least two compartments, the second compartment arranged proximal to a second, negatively charged electrode;
applying a first electrophoretic field via the first and second electrodes such that the negatively charged detergent moves to and into or through the first compartment containing the cell suspension, such that cells in the in the first compartment are lysed substantially without any viscous shear from liquid mixing;
applying a second electrophoretic field or continuing the first electrophoretic field so as to conduct electrophoresis under conditions suitable for size selection of desired ecDNA, such that the ecDNA is separated from larger chromosomal DNA molecules and travels down the gel column;
and
isolating the size selected ecDNA from the gel column.

2. The method of claim 1, wherein electrophoresis results in DNA of a size greater than 3 Mb become immobilized in the agarose gel.

3. The method of claim 2, wherein electrophoresis results in the DNA of greater than 3 Mb being immobilized in the wall of the first compartment.

4. The method of any of claims 1-3, wherein electrophoresis results in DNA having a size of less than 3 Mb traveling down the gel column.

5. The method of any of claims 1-4, wherein the DNA having a size of less than 3 Mb is isolated via electroelution.

6. The method of claim 5, wherein electroelution results in size fractions of the DNA less than 3 Mb in size being eluted to one or more elution modules of an elution cassette.

7. The method of any of claims 4-6, wherein the DNA of less than 3 Mb comprises ecDNA.

8. The method of claim 7, wherein a time period to complete electrophoresis corresponds to the size of the ecDNA.

9. The method of any of claims 1-7, wherein electrophoresis is performed between 2 and 9 hours.

10. The method of any of claims 1-6, further comprising analyzing at least one characteristic of the isolated ecDNA.

11. The method of claim 7 or 8, further comprises analyzing at least one characteristic of the isolated ecDNA.

12. The method of any of claims 1-11, further comprising enriching the ecDNA.

13. The method of any of claims 1-12, wherein the plurality of cells comprise any of animal cells, mammalian cells, and human cells.

14. The method of any of claims 1-12, wherein the plurality of cells comprise fungal cells that have been enzymatically treated to remove cell walls.

15. The method of any of claims 1-12, wherein the plurality of cells comprise plants cells.

16. The method of any of claims 1-12, wherein the plurality of cells comprise plants cells that have been enzymatically treated to remove cell walls which would otherwise prevent cell lysis by anionic detergents.

17. The method of any of claims 1-12, wherein the plurality of cells comprise bacterial cells.

18. The method of any of claims 1-12, wherein the plurality of cells comprise bacterial cells that have been enzymatically treated to remove cell walls which would otherwise prevent cell lysis by anionic detergents.

19. The method of any of claims 1-18, wherein the cells in the cells suspension are uniformly dispersed.

20. The method of any of claims 1-19, wherein the at least two compartments are arranged proximate one another.

21. The method of any of claims 1-20, wherein isolating the size selected ecDNA from the gel column is via electroelution.

22. The method of any of claims 1-21, wherein the second electrophoretic field or continuing the first electrophoretic field is applied so as to conduct electrophoresis under conditions suitable for size selection of desired ecDNA, such that the ecDNA is separated from larger chromosomal DNA molecules.

23. The method of any of claims 1-22, wherein isolating the size selected ecDNA from the gel column is via electroelution.

24. The method of any of claims 10-23, wherein the at least one characteristic selected from the group consisting of size, topology, and sequence content.

25. The method of any of claims 1-24, further comprising determining the DNA sequence of the ecDNA.

26. The method of any of claims 1-25, further comprising imaging the ecDNA.

27. The method of claim 26, wherein imaging is via at least one of: optically, electron microscopy, atomic force microscopy.

28. A method according to any one or more of the method embodiments, and/or one or more steps thereof, disclosed herein.

29. At least one of a system and a device configured to performing one or more of the methods disclosed herein.

30. An agarose gel cassette including at least two wells/cavities/compartments, for holding liquid samples positioned in relatively close proximity and configured to enable or perform one or methods of the present disclosure.
